SEQUENCE LISTING (1 sequence)

SEQ ID NO: 1 - DNA, 22 bp, Caenorhabditis elegans
  source 1..22: mol_type = genomic DNA
  modified_base 1..22: mod_base = OTHER; note = contiguous residues linked by phosphorothioate bonds
tcaccgggtgtaaatcagcttg

(19) United States
(12) Patent Application Publication
(10) Pub. No.: US 2025/0138021 A1
(43) Pub. Date: May 1, 2025

(54) OLIGOMER BARCODE FOR LABELING EXTRACELLULAR VESICLES

(71) Applicant: University of Washington, Seattle, WA (US)

(72) Inventors: Elizabeth Nance, Seattle, WA (US); Nam Phuong H Nguyen, Seattle, WA (US); Shahrokh Paktinat, Seattle, WA (US); Lucia Vojtech, Seattle, WA (US); Mengying Zhang, Seattle, WA (US)

(73) Assignee: University of Washington, Seattle, WA (US)

(21) Appl. No.: 18/930,559

(22) Filed: Oct. 29, 2024

Related U.S. Application Data
(60) Provisional application No. 63/594,885, filed on Oct. 31, 2023.

(51) Int. Cl. G01N 33/58

(52) U.S. Cl. CPC G01N 33/58

(57) ABSTRACT

This application relates to oligomer barcodes for labeling extracellular vesicles (EVs). The labeled extracellular vesicles can be used to monitor EV movement for research purposes and for the development of treatments in a variety of diseases and disorders.

### CROSS-REFERENCE TO RELATED APPLICATION

This application claims priority to U.S. Provisional Patent Application No. 63/594,885 filed on Oct. 31, 2023, which is incorporated herein by reference in its entirety as if fully set forth herein.

### STATEMENT REGARDING FEDERALLY SPONSORED RESEARCH OR DEVELOPMENT

This invention was made with government support under Grant No. R01AI153342, awarded by the National Institutes of Health. The government has certain rights in the invention.

### REFERENCE TO SEQUENCE LISTING

The Sequence Listing associated with this application is provided in XML format in lieu of a paper copy and is hereby incorporated by reference into the specification. The name of the file containing the Sequence Listing is W149-0060US-Seq.xml. The file is 2,313 bytes, was created Oct. 29, 2024, and is being submitted electronically via Patent Center.

### TECHNICAL FIELD

This application relates to oligomer barcodes for labeling extracellular vesicles (EVs). The labeled EVs can be used to monitor EV movement for research purposes and for the development of treatments in a variety of diseases and disorders.

### BACKGROUND

Extracellular vesicles (EVs) are lipid-bound nanoparticles that are released by cells in the body. EVs contain a wide repertoire of biomolecules involved in critical physiological pathways. Though the mechanisms of EV biodistribution and cell-specific uptake remain unclear, EV cargo can be transferred to neighboring or distant cells, acting as an important mode of intercellular communication. EVs are an attractive alternative to traditional synthetic nanoparticles for drug delivery due to their critical roles in cell communication, therapeutic potential, and ability to carry cargo and be trafficked to recipient cells with low immunogenicity.

### BRIEF DESCRIPTION OF THE DRAWINGS

FIG. 1 illustrates an example environment for labeling extracellular vesicles (EVs).

FIG. 2 illustrates an example process for conjugating extracellular vesicles to oligonucleotide labels.

FIGS. 3A, 3B illustrate example oligobarcode design and graphical abstract. FIG. 3A illustrates oligobarcode design with amino and phosphorothiate bond modifications to improve the stability against exonucleases in physiological conditions. FIG. 3B illustrates a schema of experimental design for oligobarcode exposure studies and polymerase chain reaction (PCR) strategy, created in BioRender (BioRender of Toronto, Canada).

FIG. 4 illustrates oligobarcode expression following stemloop procedure. Expression levels of oligbarcode using various dilutions of stem-loop primer compared to a non-stem-loop control. Annealing temperatures used are 55° C. and 60° C.

FIGS. 5A, 5B illustrate dilution series quantifying oligobarcode expression following conjugation to EVs from different biological sources. FIG. 5A illustrates a comparison of dilution series of oligobarcoded SEVs (oligo-SEVs) and BEVs (oligo-BEVs) to quantify oligo-EV expression for qPCR. Results are averaged from two technical replicates. FIG. 5B illustrates direct digital PCR results quantifying copies of oligobarcode detected per input number of SEV. Oligo-SEVs tested were from the same batch and any value below log3 of input SEV was undetected by digital PCR. Triplicates from every sample were run and measured with direct digital PCR, with each data point representing the average copy number across triplicates.

FIGS. 6A-6C illustrate example detection limits of oligo-EVs. FIG. 6A illustrates a comparison of the detection levels of oligobarcode using different DNA extraction techniques. Dilutions of oligo-SEV and oligo-BEVs were exposed to either commercial Qiagen DNA extraction columns ('Extracted DNA') or Direct PCR using heat lysis without extraction columns. To apply this technique in vitro, the qPCR detection limits of oligobarcode were compared when (FIG. 6B) different dilutions of oligo-SEV spike-in were exposed to 25,000 human THP cells and when (FIG. 6C) different dilutions of THP cells were exposed to 10⁷ molecules of oligo-SEVs. Cells were incubated with oligo-SEVs for 24 h before processing. Every point represents the average of two replicates.

FIGS. 7A-7C illustrate ex vivo application and quantification of oligo-SEVs on digested human vaginal tissue. FIG. 7A illustrates ex vivo human vaginal cells from digested tissue were exposed to oligo-SEVs and unconjugated oligo for 6 & 24 h. At the completion of the exposure period, antigen presenting cells (APCs) were isolated from non-APCs (flow through) using magnetic beads. Oligo expression (FAM) was measured with qPCR and normalized against a housekeeper gene (Late RPP30-HEX). All conditions were normalized against the level of blank oligo expression detected in APCs (negative control). N=5, bars plot median with standard deviation. FIG. 7B illustrates comparing efficacy of using preamp solution vs. without using preamp solution following 6 h of oligo-SEV exposure to ex vivo vaginal tissue. Fold changes in oligo-SEV amplification compared to non-treated (cell only) control. N=3 biological replicates. FIG. 7C illustrates evaluating cell viability of vaginal cells exposed to oligo-SEVs over 6 and 24 h using AlamarBlue metabolic assay.

FIGS. 8A, 8B illustrate ex vivo application and quantification of oligo-BEVs on digested rat brain tissue. FIG. 8A illustrates a dilution series of oligobarcode-BEV exposure to ex vivo organotypic whole hemisphere brain slices for 24 h. Oligobarcode signal was measured with fluorescent probe-based qPCR and normalized against a housekeeper gene (GAPDH). FIG. 8B illustrates representative probe-based qPCR amplification results comparing oligobarcode-BEV signal (black) to blank unconjugated oligobarcode signal (gray) in one sample. Samples with oligobarcoded BEVs demonstrated about 2¹⁵-2¹⁷ fold more signal (threshold cycle: 30-35) compared to blank oligobarcodes (threshold cycle: 47) when administered to brain slices. N=9 biological replicates, with each data point representing an average of 3 technical replicates.

FIGS. 9A-9D illustrate in vivo murine administration of oligo-SEVs and blank oligo for 24 h. Oligo-SEVs and blank oligo were vaginally administered. Oligo-BEV and blank oligobarcode expression were quantified in (FIGS. 9A, 9C) APCs and (FIGS. 9B, 8D) non-APC cells. Oligobarcode signal was normalized against the housekeeping gene RPP30 expression, and then compared against a blank PBS control. N=4-6 biological replicates for oligo-SEV and 2 biological replicates for blank oligo. Oligobarcode detection levels were compared to a blank saline control.

### DETAILED DESCRIPTION

Extracellular vesicles (EVs) are lipid-bound nanoparticles. When released by cells, they are involved in critical physiological pathways (Chand S, et al., ACS Applied Nano Materials. 2020;3(9):8906-19; Gudbergsson J M, et al., Journal of Controlled Release. 2019;306:108-20; Yáñez-Mó M, et al., Journal of Extracellular Vesicles. 2015;4 (1):27066). Though the mechanisms of EV biodistribution and cell-specific uptake remain unclear, EV cargo can be transferred to neighboring or distant cells, acting as an important mode of intercellular communication (Turturici G, et al., American Journal of Physiology-Cell Physiology. 2014;306(7):C621-C33; Tkach and Thery. Cell. 2016;164(6):1226-32). EVs are an attractive alternative to traditional synthetic nanoparticles for drug delivery due to their critical roles in cell communication, therapeutic potential, and ability to carry cargo and be trafficked to recipient cells with low immunogenicity (Witwer and Wolfram. Nature Reviews Materials. 2021;6(2):103-6; Klyachko N L, et al., Pharmaceutics. 2020;12(12):1171; Sun D, et al., Mol Ther. 2010;18(9):1606-14; El Andaloussi S, et al., Nature Reviews Drug Discovery. 2013;12(5):347-57; Cheng and Hill. Nat Rev Drug Discov. 2022;21(5):379-99). For effective translation of EVs into the therapeutic space there is a need to monitor the uptake and localization of EVs within cells and tissue to quantify EV biodistribution and behavior in physiological environments. However, monitoring EV localization within cells, tissues, and biofluids is challenging due to their small size, heterogeneity and lack of current labeling techniques that offer quantitative results (Yi YW, et al., Int J Mol Sci. 2020;21(2); Betzer O, et al., Wiley Interdiscip Rev Nanomed Nanobiotechnol. 2020;12(2):e1594). Additionally, for purposes of tracking the distribution of administered EVs within a biological model, it is difficult to distinguish endogenous EVs from those that are externally applied.

In recent years, various methods have emerged for imaging EVs to study their cellular targeting (Betzer, supra; Li Y J, et al., J Control Release. 2020;328:141-59; Almeida S, et al., Int J Mol Sci. 2020;21(24); Choi and Lee. Stem Cell Research & Therapy. 2016;7(1)). Common labeling strategies include the use of lipophilic dyes, radiolabeling, and genetic engineering. However, these labeling techniques have significant disadvantages. The gold standard labeling technique for EVs is the use of lipophilic dyes, such as Dil and PKH67, that anchor to the lipid membrane of EVs (Choi and Lee, supra; Shimomura T, et al., Bioconjug Chem. 2021;32(4):680-4). Though these dyes are commercially available and are applied using a relatively straightforward protocol, they can aggregate or leak out of membranes to produce background signal and have a low penetration depth within tissues (Shimomura, supra; Takov K, et al., J Extracell Vesicles. 2017;6(1):1388731).

In radioisotope labeling of EVs, a transmembrane protein is labeled with a radioisotope and either loaded into the EV lumen or tagged to their surface and imaged with special cameras (Li, supra; Choi and Lee, supra). Though this technique allows for imaging within deep tissue and is highly sensitive and specific, handling the radioactive material required for this technique is highly regulated. In addition, special equipment is required to image radiolabeled EVs, which is expensive and necessitates specialized training of personnel to operate. EVs can also be genetically engineered to express recombinant proteins with fluorescent tags (Richter M, et al., Advanced Drug Delivery Reviews. 2021;173:416-26; Salunkhe S, et al., J Control Release. 2020;326:599-614). However, genetic engineering can be time consuming, the fluorescent tags can be degraded by proteases within the cells, and there is limited physiological relevance of this technique.

The use of oligonucleotide barcodes (oligobarcodes) as a novel technique for labeling EVs is described herein, and addresses various problems of previous techniques. Oligobarcoding is an advantageous EV labeling technique due to its use of fast and efficient click chemistry to conjugate DNA-based biobarcodes to the surface of EVs. Various examples of oligobarcodes described herein can be readily quantified using standard analytical methods such as polymerase chain reaction (PCR) to amplify the barcode sequence(s) with high sensitivity. 4-formylbenzoate (4FB) has been previously linked to 6-hydrazinonicotinate acetone hydrazone (HyNic) click chemistry to label EVs and virions with fluorescent quantum dots and demonstrated stable, highly tailorable constructs with preservation of EV function (Zhang M, et al., Nanoscale Advances. 2019;1 (9): 3424-42). In some implementations of the present disclosure, a similar chemistry is used to conjugate a synthetic 5′ amine-containing small oligonucleotide to EVs derived from human semen and from whole rat brain tissue. For instance, some examples of the small oligonucleotide barcode are based on the sequence of C. elegans microRNA 39 to enable use of commercial stem-loop PCR assays and is modified with phosphothioate bonds to increase stability during culture with cells and tissues.

Implementations of the oligobarcoding methods described herein can be used to track EV fate from two different EV sources—semen (SEVs) and brain tissue (BEVs)—within two different models: in vitro digested vaginal tissue and ex vivo brain slices. Semen carries a high concentration of EVs (an average of 1×10¹³ per ejaculate) containing immunosuppressive biomolecules that can act to regulate the female immune system upon delivery favoring a healthy pregnancy (Kaminski V D L, et al., Heliyon. 2019;5(8); Vojtech L, et al., Nucleic Acids Research. 2014;42(11):7290-304; Vojtech L, et al., Nucleic Acids Res. 2014;42(11):7290-304). It has been shown that SEVs efficiently bind to and enter antigen-presenting cells (APCs), markedly reducing antigen-specific cytokine production (Vojtech L, et al., PLOS ONE. 2019;14(10)). EVs also maintain therapeutic functionality in the brain, as BEVs have been found to improve cell viability, increase anti-inflammatory cytokine expression, and shift microglia towards a restorative phenotype following ex vivo hypoxic ischemic injury (Heidarzadeh M, et al., Molecular Neurobiology. 2022; Zhang M, et al., ACS Applied Nano Materials. 2020;3(7):7211-22). This conjugation technique can be applied to EVs derived from any source and can be used in vitro, ex vivo, and in vivo. Implementations of the disclosed oligobarcoding methods establish a robust, accessible tool for quantifying EV association with specific cell types in cell culture and in-vivo conditions, enabling a greater understanding of EV fate and function.

Various implementations described herein relate to methods and compositions for tracking movement and cellular uptake of labeled EVs. In various implementations described herein, EVs are labeled with an oligonucleotide barcode. According to some implementations, the labeled EVs can be detected ex vivo or in vitro. Implementations of the present disclosure describe conjugation of EVs to oligonucleotide barcodes using covalent bonding. According to some implementations, the conjugation of EVs to oligonucleotide barcodes uses a primary amine. Implementations of the present disclosure include oligonucleotide barcodes conjugated on the surface of EVs. In some implementations, oligonucleotide barcodes are internalized by EVs. Implementations described herein can be used for research and drug development uses. For instance, using the techniques described herein, the uptake of a candidate therapeutic agent in cells of a host subject (e.g., an animal model) can be identified. Identifying whether the candidate therapeutic is delivered to target cells can be a step to validating the efficacy of the candidate therapeutic.

Various implementations of labeled EVs described herein are not products of nature and are not naturally occurring. For instance, implementations described herein utilize oligonucleotide sequences that are not naturally found in the organism from which the EVs are derived (e.g., isolated from). Using oligonucleotides that are not naturally found in the organism that the EVs are administered to enables distinction of the oligonucleotide labels described herein from endogenous nucleic acids in a sample collected from the organism. In various implementations of the present disclosure, the oligonucleotide labels are configured to avoid degradation by nucleases. For instance, the oligonucleotide labels are modified to include one or more phosphorothioate bonds. Accordingly, various implementations of the oligonucleotide labels described herein are not products of nature and are not naturally occurring.

Particular examples will now be described with reference to the accompanying figures. The scope of this disclosure includes individual examples described herein as well as any combination of the examples, unless otherwise specified.

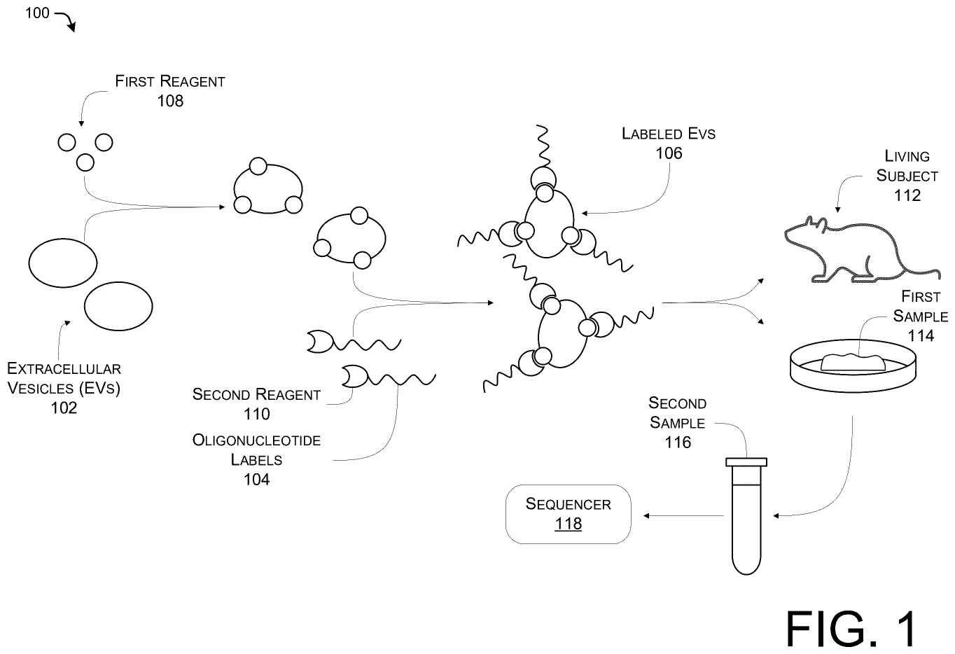

FIG. 1 illustrates an example environment 100 for labeling extracellular vesicles. Extracellular vesicles (EVs) 102 are, in some examples, isolated from a subject or from cultured cells. The subject, in various cases, may be a mouse, a rat, a mammal, a non-human primate, human, or another organism. The cultured cells, in various cases, are derived from kidney cells (e.g., HEK293 cells, HK-2 cells, A498 cells, MDCK cells, LLC-PK1 cells, etc.), lung cells (A549 cells, BEAS-2B cells, H460 cells, Calu-3 cells, L132 cells, WI-26 cells, etc.), heart cells (AC16 cells, H9c2 cells, etc.), muscle cells (RD cells, SAT2 cells, L6 cells, C2C12 cells), cervical cells (e.g., HeLa cells, C33a cells, SiHa cells, CaSki cells, Z310 cells), pancreatic cells (e.g., PANC-1 cells, MIA PaCa-2 cells, AsPC-1 cells, Capan-1 cells, HPNE cells, INS-1 cells, etc.), epithelial cells (e.g., Hela cells, A549 cells, Caco-2 cells, T84 cells, Calu-3 cells, etc.), adrenal cells (e.g., H295R cells, SW-13 cells, COS-1 cells, etc.), brain cell (e.g., SH-SY5Y cells, U87MG cells, A172 cells, Neuro2a cells, C6 cells, etc.), bladder cells (T24 cells, RT4 cells, J82 cells, UROtsa cells, 5637 cells, etc.), immune cells (e.g., Jurkat cells, Ramos cells, THP-1 cells, MOLT-4 cells, K562 cells, MCF-7 cells, U937 cells, etc.), colorectal cells (e.g., Caco-2 cells, HT-29 cells, T84 cells, SW480 cells, LIM 1215 cells, RKO cells, etc.), gastric cells (e.g., AGS cells, MKN45 cells, KATO III cells, SNU-1 cells, NCI-N87 cells, GEC cells), stem cells (e.g., H9 cells, H1 cells, NSC-34 cells, etc.), or any other suitable cell type. In some examples, the cultured cells include cells isolated directly from a living subject, such as primary cardiomyocytes, primary hepatocytes, primary endothelial cells, primary fibroblasts, primary adipocytes, primary myoblasts, primary neurons, primary stem cells, human umbilical vein endothelial cells, and the like.

In some examples, the EVs 102 are modified. For instance, the surface of the EVs may be modified to include targeting agents configured to bind to specific cell types or biomarkers (e.g., disease-specific biomarkers). In some examples, the EVs 102 may be derived from (e.g., released by) chimeric antigen receptor (CAR)-T cells that are configured to bind specific cell types (e.g., cancer cells). In various implementations, the cargo of the EVs 102 may be modified. For instance, the EVs may be loaded with nucleic acids, gene editing reagents, proteins, small molecules, or the like.

In various implementations, it may be beneficial to track the movement of EVs within a space, such as an organism. For example, EVs can be used as delivery vehicles for therapeutic agents. Accordingly, characterizing their movement can facilitate more effective delivery of therapeutic agents. In some examples, migration patterns of EVs in a subject may change due to a pathological condition of the subject. Examples of pathological conditions include cancer, autoimmune diseases, inflammatory diseases, infectious diseases, cardiac diseases, neurodegenerative diseases, respiratory diseases, and other diseases. In some examples, pathological conditions may include abnormal physiological conditions of the subject due to, for instance, an injury, a wound, stress, exercise, a mental health condition, vitamin or mineral deficiency, or the like. For instance, behavior of EVs responsible for activation of the immune system may change due to the progression of an autoimmune disease in the subject. Accordingly, understanding the migration patterns may facilitate understanding of disease pathogenesis as well as identification of new therapeutic and/or preventative targets. EVs, in various cases, are labeled with fluorescent or radioactive labels. However, these labels are difficult to detect due to the reliance on expensive microscopes and highly trained users. Due to the size of these labels, it can be difficult to identify single labels, thus limiting the quantification ability of microscopy. In addition, the reliance on microscopy can limit sensitivity and quantification, for instance, due to background fluorescence from light scattering, auto-fluorescence, intrinsic protein fluorescence, and other factors. In addition, radioactive labels (e.g., radioisotopes) can be difficult to handle due to radiation safety precautions, including personal protective equipment, radiation monitoring, training, etc.

In various implementations of the present disclosure, these issues can be addressed by using labels that can be amplified, enabling more sensitive detection, and detected without highly trained users and strict safety precautions. In various implementations, the labels include oligonucleotide labels 104. The oligonucleotides can be amplified using, for instance, polymerase chain reaction (PCR), isothermal amplification (e.g., loop-mediated isothermal amplification (LAMP), nucleic acid sequence-based amplification (NASBA), multiple displacement amplification (MDA), etc.), or another suitable amplification technique. Amplification can, in some examples, enable the identification of a single oligonucleotide in a sample. Many laboratories, including some in low-resource settings, include or have access to genomic sequencing equipment due to the rise of sequencing-based diagnostic tests. Accordingly, utilizing sequencing, rather than microscopy, can improve the accessibility of various implementations described herein.

According to some implementations, the EVs 102 are conjugated (e.g., linked) to the oligonucleotide labels 104 to form labeled EVs 106. As used herein, the term “oligonucleotide label,” and its equivalents, may refer to a short nucleic acid (e.g., DNA or RNA). Oligonucleotide labels, in various examples, include a nucleic acid with a length less than 100 nanometers (nm). According to some implementations, oligonucleotide labels are detectable. For instance, oligonucleotide labels may include a phosphorothioate bond to prevent degradation by nucleases. In some examples, oligonucleotide labels are configured to attach to a linker, such as a linker configured to bind to particle (e.g., an EV). For example, oligonucleotide labels may include a primary amine to facilitate conjugation to a click chemistry reagent.

In various cases, the EVs 102 are linked to a first reagent 108 that is configured to bind to a second reagent 110 linked to the oligonucleotide labels 104. In some instances, the oligonucleotide labels 104 include a 5′ primary amine that facilitates conjugation to the second reagent 110. The first reagent 108 is configured, in some examples, to covalently bind to the second reagent 110. In particular examples, the first reagent 108 and the second reagent 110 are click chemistry reagents that are configured to selectively bind with high efficiency. The term “click chemistry,” “click chemistry reactions,” and their equivalents, may refer to reactions that are regiospecific (e.g., that produce one structural isomer), provide high chemical yields (e.g., a yield above 80%), are insensitive to oxygen and water, and produce a product that is stable in physiological conditions. In some implementations, click chemistry reactions do not include a solvent or include a solvent that can be easily removed (e.g., water). In some implementations, click chemistry reactions do not include purification to isolate the product or include nonchromatographic purification methods, such as crystallization or distillation. The first reagent 108, in some examples, comprises 4-formylbenzoate (4FB), and the second reagent 110 comprises 6-hydrazinonicotinate acetone hydrazone (HyNic). In various cases, the conjugation does not include the use of a metal catalyst. Metal catalysts can be toxic to living subjects and cells, and may not be suitable for biomedical applications.

In some examples, the oligonucleotide labels 104 are conjugated to the EVs 102 at a ratio in a range of 1 EV:10 oligonucleotide labels to 1 EV:100 oligonucleotide labels. In some implementations, the oligonucleotide labels 104 are conjugated to the EVs 102 at a ratio of 1 EV:80 oligonucleotide labels.

The oligonucleotide labels 104, in various cases, include a nucleic acid sequence derived from an organism that is different than the living subject 112 (e.g., a nucleic acid sequence that is not endogenous to the living subject 112). For instance, the living subject 112 may be a mouse, a rat, a mammal, a non-human primate, or a human, and the oligonucleotide labels 104 may include a sequence derived from C. Elegans. In various examples, the oligonucleotide labels 104 may include one or more sequences derived from bacteria, yeast, a nematode, an insect (e.g., fruit flies), an amphibian, a mouse, a rat, a non-human primate, a human, a plant (e.g., Arabidopsis, Amborella trichopoda, Arabidopsis lyrata, Arachis hypogaea, Asparagus officinalis, Brassica napus, Brassica rapa, Carica papaya, Citrus sinensis, Cucumis melo, Fragaria vesca, Glycine max, Helianthus tuberosus, Hevea brasiliensis, Malus domestica, Manihot esculenta, Medicago truncatula, Nicotiana tabacum, Phaseolus vulgaris, Populus trichocarpa, Prunus persica, Ricinus communis, Solanum lycopersicum, Vitis vinifera, Vriesea carinata), or any other living organism. The oligonucleotide labels 104 may include DNA and/or RNA sequences. In some examples, a length of the oligonucleotide labels 104 is in a range of 10 to 80 nucleotides. In various implementations, a length of the oligonucleotide labels 104 is in a range of 15 to 35 nucleotides. According to some examples, the oligonucleotide labels 104 may be modified to improve stability and/or resistance to nuclease degradation. For instance, the oligonucleotide labels 104 may include one or more phosphorothioate bonds. In particular examples, the oligonucleotide labels 104 may be linked to the EVs 102 by electroporation or lipofection.

In some examples, the oligonucleotide labels 104 include one or more sequences. For instance, the oligonucleotide labels 104 may include first oligonucleotides with a first sequence and second oligonucleotides with a second sequence. The first sequence is distinct from the second sequence, for instance. The first oligonucleotides may be configured to label a first EV type of the EVs 102, and the second oligonucleotides may be configured to label a second EV type of the EVs 102. For instance, the first oligonucleotides may label exosomes in the EVs 102, and the second oligonucleotides may label microvesicles in the EVs. EV types include exosomes, microvesicles, apoptotic bodies, autophagic EVs, matrix vesicles, and stressed EVs. In various implementations, EV types include organ-specific EVs, such as pulmonary EVs, cardiovascular EVs, brain EVs, hepatic EVs, pancreatic EVs, renal EVs, gastrointestinal ECs, and the like. In some cases, EV types include cell type-specific EVs, such as epithelial EVs, endothelial EVs, eosinophil EVs, mast cell EVs, cancer-derived EVs, and the like.

In various examples, the labeled EVs 106 are administered to a living subject 112 or to a first sample 114 derived from the living subject 112. The living subject 112 is, in some cases, a mouse, a rat, a mammal, a non-human primate, human, or another organism. In some implementations, the EVs 102 may be isolated from the living subject 112, a different subject, or from cultured cells. In various instances, the sample is a tissue sample (e.g., a tissue biopsy) or a liquid sample (e.g., a urine sample, a cerebrospinal fluid (CSF) sample, a milk sample, a semen sample, a blood sample, a saliva sample, or the like). In various cases, the EVs 102 may be isolated from a fresh sample or a stored sample. For instance, the sample may be cryopreserved or preserved using a fixative. In various example, the cells of the sample may be cultured before the EVs 102 are isolated.

In some cases, the labeled EVs 106 can be administered to the living subject 112 by, for example, injection, inhalation, infusion, perfusion, lavage, or ingestion. Routes of administration can include intravenous, intradermal, intraarterial, intranodal, intravesicular, intrathecal, intraperitoneal, intraparenteral, intranasal, intralesional, intramuscular, intravaginal, rectal, oral, subcutaneous, topical, and/or sublingual administration. Formulations are generally administered by injection. In some cases, the labeled EVs 106 are applied to the first sample 114, for instance, by direct application, immersion, incubation, or the like.

In some implementations, the labeled EVs 106 are administered with a pharmaceutically acceptable carrier. Exemplary pharmaceutically acceptable carriers and formulations are disclosed in Remington's Pharmaceutical Sciences, 18th Ed. Mack Printing Company, 1990. Moreover, formulations can be prepared to meet sterility, pyrogenicity, general safety and purity standards as required by United States FDA Office of Biological Standards and/or other relevant foreign regulatory agencies.

Exemplary generally used pharmaceutically acceptable carriers include any and all bulking agents or fillers, solvents or co-solvents, dispersion media, coatings, surfactants, antioxidants (e.g., ascorbic acid, methionine, vitamin E), preservatives, isotonic agents, absorption delaying agents, salts, stabilizers, buffering agents, chelating agents (e.g., EDTA), gels, binders, disintegration agents, and/or lubricants. Adsorption delaying agents include controlled release formulations, such as coatings, water-insoluble encapsulations, matrices (e.g., polymer matrices, lipid formulations, hydrogels, ion-exchange resins, cross-linked gels, etc.), and the like.

Exemplary buffering agents include citrate buffers, succinate buffers, tartrate buffers, fumarate buffers, gluconate buffers, oxalate buffers, lactate buffers, acetate buffers, phosphate buffers, histidine buffers and/or trimethylamine salts.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol and 3-pentanol.

Exemplary isotonic agents include polyhydric sugar alcohols including trihydric or higher sugar alcohols, such as glycerin, erythritol, arabitol, xylitol, sorbitol or mannitol.

Exemplary stabilizers include organic sugars, polyhydric sugar alcohols, polyethylene glycol, sulfur-containing reducing agents, amino acids, low molecular weight polypeptides, proteins, immunoglobulins, hydrophilic polymers or polysaccharides.

In particular implementations, treatments described herein (e.g., the labeled EVs 106) can be formulated into a carrier in a therapeutically-effective amount. As described herein, exemplary carriers include saline, buffered saline, physiological saline, water, Hanks' solution, Ringer's solution, Normosol-R (Abbott Labs), PLASMA-LYTE A® (Baxter Laboratories, Inc., Morton Grove, IL), and combinations thereof.

Compositions including the labeled EVs 106 and/or other components described herein may be administered in a formulation that includes one or more carriers, stabilizers, anesthetics, preservatives, or any combinations thereof.

In particular implementations, carriers can be supplemented with human serum albumin (HSA) or other human serum components or fetal bovine serum. In particular implementations, a carrier for infusion includes buffered saline with 5% HSA or dextrose. Additional isotonic agents include polyhydric sugar alcohols including trihydric or higher sugar alcohols, such as glycerin, erythritol, arabitol, xylitol, sorbitol, or mannitol.

Carriers can include buffering agents, such as citrate buffers, succinate buffers, tartrate buffers, fumarate buffers, gluconate buffers, oxalate buffers, lactate buffers, acetate buffers, phosphate buffers, histidine buffers, and/or trimethylamine salts.

Stabilizers refer to a broad category of excipients which can range in function from a bulking agent to an additive which helps to prevent cell adherence to container walls. Typical stabilizers can include polyhydric sugar alcohols; amino acids, such as arginine, lysine, glycine, glutamine, asparagine, histidine, alanine, ornithine, L-leucine, 2-phenylalanine, glutamic acid, and threonine; organic sugars or sugar alcohols, such as lactose, trehalose, stachyose, mannitol, sorbitol, xylitol, ribitol, myoinisitol, galactitol, glycerol, and cyclitols, such as inositol; PEG; amino acid polymers; sulfur-containing reducing agents, such as urea, glutathione, thioctic acid, sodium thioglycolate, thioglycerol, alpha-monothioglycerol, and sodium thiosulfate; low molecular weight polypeptides (e.g., <10 residues); proteins such as HSA, bovine serum albumin, gelatin or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran.

Where beneficial, formulations can include a local anesthetic such as lidocaine to ease pain at a site of injection.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol, and 3-pentanol. In various implementations, the labeled EVs 106 are administered to the living subject 112 without a stabilizer and/or a solvent.

In various implementations, the labeled EVs 106 are detected from the living subject 112 or the first sample 114. For instance, the labeled EVs 106 may be administered to the living subject 112, and at least one second sample 116 (e.g., a tissue biopsy sample, a liquid biopsy sample, or the like) may be collected from the living subject 112. In some examples, the second sample(s) 116 includes at least a portion of the first sample 114. Each of the second sample(s) 116, in various instances, may be collected from a spatially distinct portion of the living subject 112 or of the first sample 114. In various cases, the second sample(s) 116 may be processed to facilitate detection of the labeled EVs 106. For instance, cells (e.g., of the living subject 112 or of the first sample 114) in the second sample(s) 116 may be processed, for example, by lysing the cells, extracting nucleic acids from the cells (e.g., by phenol-chloroform extraction, solid-phase extraction, magnetic bead-based purification, etc.), or the like. The second sample(s) 116 may be, in various cases, applied to a sequencer 118 that is configured to detect the sequence(s) of the oligonucleotide labels 104 of the labeled EVs 106.

The sequencer 118, in various implementations, is configured to amplify nucleic acids and/or detect sequence(s) of the amplified nucleic acids (e.g., by polymerase chain reaction (PCR), isothermal amplification, unbiased DNA sequencing, or the like). Amplification, in some examples, includes repeated cycles of denaturation of nucleic acids, annealing the nucleic acids to primers, and extension of the nucleic acids using a polymerase. In some examples, the amplification does not include temperature cycling. For instance, the nucleic acids may be amplified using loop-mediated isothermal amplification (LAMP), recombinase polymerase amplification (RPA), or the like. The sequences of the nucleic acids, in various instances, can be detected by gel electrophoresis, intercalating dyes, detectable probes configured to hybridize to the oligonucleotide labels 104, adapters configured to ligate to nucleic acids, or the like.

In some examples, the sequencer 118 is configured to extend the oligonucleotide labels 104 before detection. In various implementations, a length of the oligonucleotide labels 104 may limit the sensitivity of detection. Accordingly the sequencer 118 may be configured to perform stem loop amplification using a stem loop primer to extend the oligonucleotide labels 104.

In various cases, the sequencer 118 is configured to detect the labeled EVs 106 by imaging the living subject 112, the first sample 114, or the second sample(s) 116. For instance, the sequencer 118 may be configured to facilitate detection of the labeled EVs 106 using fluorescence in situ hybridization (FISH), DNA scope, RNA scope, or the like. In some examples, the cells of the first sample 114 or the second sample(s) 116 may be fixed onto a slide, for instance, by using formaldehyde or paraformaldehyde to preserve the cellular structure. In some examples, the cells may be permeabilized, for example, by applying a detergent (e.g., Triton X-100, Tween-20, digitonin, saponin, etc.) to facilitate intracellular transport of a detectable label. A detectable label that is configured to bind to the oligonucleotide labels 104 may be applied to the first sample 114 or the second sample(s) 116. In various instances, the detectable label may include an oligonucleotide, a peptide nucleic acid (PNA), a small molecule, or a protein configured to hybridize to the oligonucleotide labels 104. In various instances, the detectable includes a fluorescent dye, a fluorescent protein, a radioisotope, a quantum dot, or the like. In various examples, the detectable label may be applied to the living subject 112, the first sample 114, or the second sample(s) 116. In various cases, a second detectable label that is configured to hybridize to the detectable label may be applied to the living subject 112, the first sample 114, or the second sample(s) 116 to improve the sensitivity and/or resolution of detection. The sequencer 118 may image (e.g., by fluorescence microscopy, positron emission tomography (PET), single photon emission computed tomography (SPECT)) the living subject 112, the first sample 114, or the second sample(s) 116 to detect the labeled EVs 106. In various implementations, the sequencer 118 is configured to output an indication of a presence, a level (e.g., a concentration, an amount, etc.), or a distribution of the labeled EVs 106.

In various implementations, the oligonucleotide labels 104, the EVs 102, the labeled EVs 106, and other elements described herein can be included in one or more kits. Kits can include various components to practice methods disclosed herein. For example, kits can include the oligonucleotide labels 104, the EVs 102, the labeled EVs 106, cell lysis reagents, nucleic acid extraction reagents, sample purification reagents, amplification reagents (e.g., primers, DNA polymerases, deoxynucleotide triphosphates, etc.), detection reagents (e.g., fluorescent probes, antibodies, detectable enzymes, chemiluminescent substrates, fluorescent dyes, colorimetric dyes, molecular beacons, etc.), buffers (e.g., sample buffers, wash buffers, transfer buffers, blocking buffers, saline, buffered saline, phosphate buffered saline (PBS), etc.), Hanks' solution, Ringer's solution, tissue samples (e.g., specimens, or other organ, and/or cells derived therefrom), culture-initiating compositions, RPMI medium, non-essential amino acids, sodium pyruvate, penicillin/streptomycin, human serum albumin (HSA) or other human serum components, fetal bovine serum, dextrose, stabilizers, preservatives, culture vessels, culture plates, or the like.

In particular implementations of the present disclosure, the EVs 102 may be isolated from cultured human cells. In some examples, the EVs 102 may be modified, such as with a targeting agent (e.g., a surface-bound protein) configured to cause the EVs 102 to migrate to lung cancer cells. The EVs may be attached to the first reagent 108, and then incubated with the oligonucleotide labels 104 that are attached to the second reagent 110. In various implementations, the oligonucleotide labels 104 include an RNA sequence derived from C. Elegans. The labeled EVs 106 are administered to a first human subject 112 who has lung cancer and a human subject who does not have cancer. In various examples, a tissue biopsy may be collected from the first human subject 112 and the second human subject to determine the presence of the labeled EVs 106. For instance, tissue biopsies may be collected from the lungs of the first human subject 112 (e.g., from a lung tumor of the first human subject 112) and second human subject. The second samples 116 may be collected from the first human subject 112 and second human subject. The labeled EVs 106 may be detected from the tissue biopsies by applying the second samples 116 to the sequencer 118 (e.g., to perform amplification, such as PCR-based amplification, and/or sequencing). In various examples, a higher level of the labeled EVs 106 may be present in the first human subject 112 than in the second subject. Accordingly, the EVs 102 may be identified as promising drug delivery vehicles for cancer treatment. In some examples, the EVs 102 may be identified as targets for cancer treatment, for instance, to reduce inflammation in the tumor microenvironment.

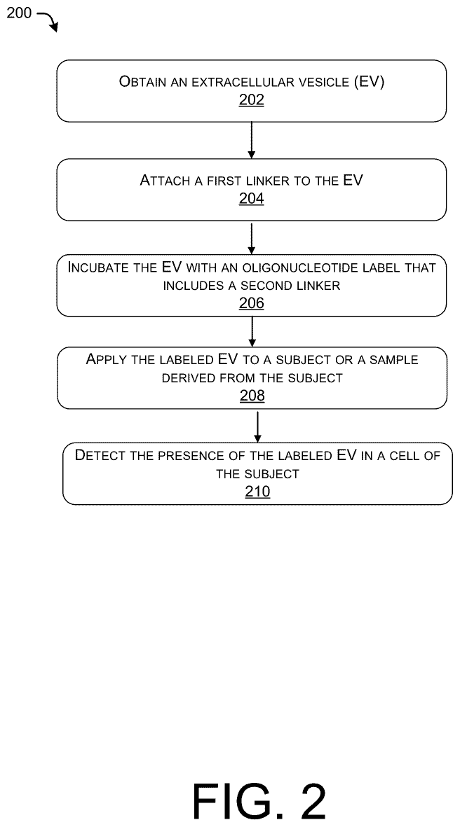

FIG. 2 illustrates an example process 200 for conjugating extracellular vesicles (e.g., the EVs 102) to oligonucleotide labels (e.g., the oligonucleotide labels 104). The process 200 is performed by an entity, which may include one or more of a fluidic device (e.g., a microfluidic device), cell culture equipment (e.g., cell culture plates, incubators, etc.), a laboratory equipment (e.g., an orbital shaker, a centrifuge, a polymerase chain reaction (PCR) machine, a thermocycler, a heating block, a microscope, etc.) a computing device, or a user (e.g., a laboratory technician, care provider, or the like). According to some implementations, any of the steps of process 200 may be omitted.

At 202, the entity obtains an EV. The EV may be isolated from an organism or from cultured cells. At 204, the entity attaches a first linker (e.g., the first reagent 108) to the EV. In some examples, the first linker is a click chemistry reagent. In various cases, the first linker is configured to attach to a functional group present on the surface of the EV. For example, the first linker may be configured to attach to an amine group, a thiol group, a carboxylic group, or another group on the surface of the EV.

At 206, the entity incubates the EV with an oligonucleotide label that includes a second linker (e.g., the second reagent 110), thereby generating labeled EVs (e.g., the labeled EVs 106). In various cases, the second linker is a click chemistry reagent configured to react with the first linker to produce a click chemistry reaction. In some examples, the first linker includes hydrazinonicotinamide (HyNic), and the second linker includes 4-formylbenzamide (4FB).

At 208, the entity applies the labeled EV to a subject (e.g., the living subject 112) or a sample (e.g., the first sample 114) derived from the subject. In some examples, the labeled EV is administered to the subject, for instance, by intravenous injection. In various cases, the labeled EV are directly applied to the sample derived from the subject. The subject, according to some examples, belongs to the same species as the organism or the organism from which the cultured cells are derived.

At 210, the entity detects the presence of the labeled EV in at least one cell of the subject. In various implementations, the entity collects a second sample (e.g., the second sample 116) from the subject. In some examples, the second sample includes a portion of the sample derived from the subject. The entity may process the second sample, such as, by lysing cells in the second sample and/or by extracting nucleic acids from cells in the second sample. The second sample is, in some cases, applied to a sequencer (e.g., the sequencer 118) that is configured to detect the presence of the oligonucleotide labels based on, for instance, the sequence of the oligonucleotide labels

Implementations of the present disclosure will now be described with reference to an Experimental Example.

### EXPERIMENTAL EXAMPLE

### Materials and Methods

Experimental Design. The objective of this Experimental Example was to develop a novel oligonucleotide barcoding technique for labeling EVs that is low-cost, quantitative, and sensitive. The DNA barcodes were covalently conjugated to the surface of semen and brain tissue-derived EVs using click chemistry. For in vitro and ex vivo studies, various dilutions of oligo-EVs were applied to cells or tissue culture for up to 24 hours (h) prior to measuring the limit of detection for oligobarcode expression using quantitative polymerase chain reaction (qPCR). Following successful oligobarcode detection both in vitro and ex vivo, oligo-SEVs were applied in vivo to the vaginal canal of murine models for 24 h. Lymph node (iliac, mesenteric, and inguinal) and genital tract (upper and lower) tissues were harvested, digested, and antigen presenting cells (APCs) were separated from the tissue cell suspensions using magnetic immunobead isolation. Oligobarcode expression within different cell populations were detected with qPCR.

Animal care and ethics. This Experimental Example was performed in accordance with the guide for the care and use of laboratory animals of the National Institutes of Health (NIH). All animals were handled according to an approved Institutional Animal Care and Use Committee (IACUC) protocol (#4383-02) of the University of Washington (UW), Seattle, WA. The UW has an approved Animal Welfare Assurance (#A3464-01) on file with the NIH Office of Laboratory Animal Welfare, is registered with the United States Department of Agriculture (certificate #91-R-0001), and is accredited by AAALAC International. Time-mated pregnant female Sprague-Dawley rats (virus antibody-free CD® (SD) IGS, Charles River Laboratories, Raleigh, NC, USA) were purchased and arrived on postnatal day 5 with a litter of 10, sex-balanced pups. Dams were housed individually with their litter and allowed to acclimate to their environment. Before and after the experiment, each dam and her pups were housed under standard conditions with an automatic 12 h light/dark cycle, a temperature range of 20-26° C., and access to standard chow and autoclaved tap water ad libitum. The pups were checked for health daily.

Isolation of semen-derived EVs (SEVs). SEV were purified from diluted semen by ultracentrifugation at 100,000 times gravity (xg) over 25% sucrose cushions for 12 h, then washed with phosphate buffered saline (PBS) and concentrated in 100 kilodalton (kDa) spin filters. SEV were characterized by nanoparticle tracking, electron microscopy, and western blotting.

Isolation of brain-derived EVs (BEVs). BEVs were isolated from whole, perfused neonatal rat brains extracted from male postnatal day 10 (P10) rats. Brain tissue was finely chopped in a solution and incubated for 20 minutes in a water bath with protease inhibitors to allow for complete dissociation of extracellular matrix proteins. Subsequently, the homogenate was initially spun at 300×g for 5 minutes, transferred to a new tube to spin at 2000×g for 10 minutes, and finally transferred to new tubes and ultracentrifuged for 10,000×g for 35 minutes. After ultracentrifugation the collected supernatant was run through an Amicon ultrafiltration column (100 kDa molecular weight cutoff; Amicon, now Millipore Sigma of Burlington, MA) and spun at 3214×g for 90-120 minutes, or until the final volume reached 500 μL. A size exclusion chromatography column (iZon of Medford, MA) was used to further purify BEVs, and fractions containing high concentrations of BEVs were ultracentrifuged in an Amicon ultrafiltration column (50 kDa) at 3214×g for 60-100 minutes at 4° C. to concentrate isolated BEVs (Nguyen N P, et al., International Journal of Molecular Sciences. 2022;23(2):620).

Conjugation of oligobarcodes to EVs (Oligo-EV). Working dilutions of hydrazinonicotinamide (HyNic), 4-formylbenzamide (4FB), and oligos were prepared in 1×PBS. Concentrated SEV and BEV stocks were added to the HyNic working solution (SEV-HyNic), while oligobarcodes were added to 4FB working solution (oligo-4FB). EV-HyNic and oligo-4FB solutions were incubated in the dark at room temperature on a rotating stand for 2 hours. After incubation, oligo-4FB was purified with a NAP-5 column (GE Healthcare of Chicago, IL) to remove excess unconjugated Sulfo-S-4FB. After 2 hours, EV-HyNic was purified using an ultrafiltration unit with a 100 kDa molecular weight cut-off (Amicon). The unit was ultracentrifuged at 3214×g to remove excess unconjugated Sulfo-S-HyNic for 30 minutes.

Purified oligo-4FB and EV-HyNic were combined at an 80:1 oligo: EV ratio and diluted to a total volume of 1 mL in 1×PBS. This sample was left in the dark on a rotating stand for 2 hours to ensure reaction between oligo-4FB and EV-HyNic. A size exclusion chromatography (SEC) column was used to separate fully conjugated oligo-SEVs from unconjugated oligo-4FB and fractions 7-9 (2.5 mL total) were collected. A control sample containing unconjugated oligo-4FB (oligo alone) with 1×PBS was also purified at the same time to ensure that the conjugation procedure did not introduce any artifacts. A final oligo-SEV concentration was performed using an Amicon 50 kDa molecular weight cut-off ultrafiltration unit, with centrifugation at 3215×g for 45 minutes.

In vitro oligo-SEV exposure experiments to vaginal cells and tissues. Human vaginal tissues were trimmed to remove excess stroma, then cut into small (30-50 mm squares) using sterile razor blades. Pieces were dissociated in freshly made collagenase (700 collagen units/mL) digestion media containing DNase (50 Units/μL) in a 37° C. orbital incubator at 200 rotations per minute (rpm) for 45 minutes. Tissue homogenate was passed through a 70 μm cell strainer to remove undigested chunks and large terminally differentiated epithelial cells, and flow through cells enriched for leukocytes were collected in a 50 ml conical tube. The isolated cells were the centrifuged at 300×g for 12 minutes at 4° C. After centrifugation, the supernatant was removed and replaced with fresh R10 media to resuspend the cell pellet. This process was repeated multiple times until all biopsy fractions were dissociated and strained. Following the last centrifugation step, isolated cells were put in R10 media and plated at a desired cell concentration in a flask or 6 well cell culture plate.

Oligo-SEVs were then administered directly to isolated cells and incubated at 37° C. for 6 and 24 h Following exposure, APCs were separated with magnetic bead isolation (EasySep Cell Separation; STEMCELL Technologies of Vancouver, Canada) against CD11c, and both CD11c+ cells and non-APC remaining cell fractions (flow through) were collected to be analyzed.

Preparation of ex vivo organotypic whole hemisphere brain slices. Following intraperitoneal euthanasia with pentobarbitol, fresh brain tissue was rapidly extracted from P10male rats, placed in ice cold dissection media, and sectioned into 300 μm thick slices using a Mcllwain tissue chopper (Ted Pella of Redding, CA). These slices were plated onto 30 mm cell culture inserts (CellTreat) and incubated at 37° C. in 1 mL of 25% slice culture medium (SCM) and 5% CO₂ to recover from acute slicing. After 24 h, oligo-BEVs were topically applied to the brain slices (109 molecules of oligo-BEVs per slice), which were then returned to 25% SCM for another 24 h before processing.

Brain slices were collected and manually digested in a Hibernate-E buffer (BrainBits of Springfield, IL) containing collagenase and protease and phosphotase inhibitors. Following digestion, samples were spun down at 450×g for 10 minutes to pellet cells prior to Direct polymerase chain reaction (PCR) and stemloop conversion.

Stemloop conversion reaction. Cell pellets were lysed in DNAse free H₂O and immediately heated at 95° C. for 5 minutes. Stemloop conversion samples contained 0.2 μL DNA polymerase (Amplitaq; Applied Biosystems of Foster City, CA), 3 μL 4X stemloop primer, 1.5 μL 10X PCR Buffer I mix, 0.5 μL 10 mM deoxynucleotide triphosphates (dNTPs), 1 μL DNAse free H₂O, and 9 μL of each sample. Samples were run with the following thermocycler settings: 1) 95° C. for 5 minutes, 2) 16° C. for 30 minutes, 3) 65° C. for 5 minutes, 4) 72° C. for 5 minutes, 5) 95° C. for 2 minutes, 6) 16° C. for 5 minutes, 7) 65°° C. for 5 minutes, and 8) 72°° C. for 5 minutes.

Direct PCR for oligo amplification. Following stemloop extension, samples oligos and gDNA targets were pre-amplified using a preamplification reagent (Taqman PreAmp Master Mix; Thermo Fisher of Waltham, MA). 20X oligobarcode primer-probe mix (fluorescein amidite (FAM)) and late ribonuclease P protein subunit p30 (RPP30) genomic control (hexachlorofluorescein (HEX)/cyanine5 (Cy5)) were diluted in tris-ethylenediaminetetraacetic acid (TE) buffer to a final concentration of 0.2X. 1.25μL of first-step PCR product was combined with 6.25 μL preamp master mix, 1.875 μL of each diluted primer-probe mix, and 1.25 μL of DNAse free H₂O. Thermocycler conditions used were: 1) 95° C. for 10 minutes, 2) 55° C. for 2 minutes, 3) 72° C. for 2 minutes, 4) 12 cycles of 95° C. for 15 seconds and 60° C. for 4 minutes, and 5) 99.9° C. for 10 minutes.

qPCR for oligo-EV target quantification. The pre-amplification products were diluted 1:10 in TE buffer to be run with qPCR. For a 20 μL total reaction volume per well, 10 μL of Taqman Proamp Master Mix was combined with 1 μL of each 20X primer-probe mix, 8 μL DNAse free H₂O and 1 μL of diluted pre-amplification sample. Thermocycler conditions used were: 1) denaturing at 95° C. for 20 seconds, followed by 2) annealing/extension for 45 cycles of 95° C. for 1 second and 65° C. for 25 seconds. Samples were run with a multi-channel setting to collect fluorescent signals concurrently from the oligobarcode (FAM) and genomic DNA (HEX) probes. Captured oligobarcode fluorescence signals were normalized by genomic DNA signals prior to calculating fold changes between conditions.

Cell viability experiments with alamarBlue. The viability and proliferation of THP cells following oligo-SEV exposure was quantified using an alamarBlue cell viability assay (Thermo Fisher). This assay uses a reagent that produces a highly fluorescent compound following its reduction by living cells. This fluorescence can be quantified to measure cell viability and proliferation, with positive absorbance unit values corresponding to positive cell viability and proliferation. 15,000 THP cells were exposed to various dilutions of oligo-SEV in a 96-well tissue treated plate. Cells were suspended with R10 media to a final volume of 100 μL and 10 μL of alamarBlue reagent was added to the cell suspension. Cells were incubated with the alamarBlue reagent at 37° C. for 6 and 24 h. At the 6 h timepoint, a plate reader was used to measure absorbance at 570 nanometers (nm) wavelength prior to media change. Fresh R10 media was added at 100 μL per well and 10 μL of alamarBlue reagent was added and incubated with cells until the 24 h timepoint. A maximum cell death sample and a media only sample were used as controls.

In vivo murine application of oligobarcode-SEVs. Female C57BL/6J mice were used for intravaginal application of oligo-SEVs. All mice were 12-16 weeks old, and injected with depo-provera 7 days prior to oligo-SEV application. For every litter of 12 mice, 6 mice were administered oligo-SEVs, 3 mice were administered with blank unconjugated oligobarcodes, and 3 were used as saline controls. Prior to administration of oligo-SEVs, mice vaginal canals are swabbed to open up the cavity and remove mucus. One dose of oligo-SEVs (1.3-3.3×1010 molecules of oligo-SEV) in a 20 μL volume were administered per mouse. Vaginal administration of oligo-SEVs, blank oligobarcode, and saline was performed 24 h following depo-provera injection, and the mice were left for an additional 24 h following the procedure for oligo-SEV uptake. Following the 24 h oligo-SEV incubation period, the mice were sacrificed and the upper and lower genital tracts as well as the iliac, mesenteric, and inguinal lymph nodes were extracted. For every condition (oligo-SEV, blank oligo, saline control) organs from 3 mice were pooled together to improve oligobarcode detection. These harvested tissues were immediately digested, immunoseparated into APC and flow through cell populations, and Direct PCR was performed for each sample.

Institutional Review Board statement. This study was performed in accordance with the guide for the care and use of laboratory animals of the National Institutes of Health (NIH). All animals were handled according to an approved Institutional Animal Care and Use Committee (IACUC) protocols (#4383-02) of the University of Washington (UW), Seattle, WA. The UW has an approved Animal Welfare Assurance (#A3464-01) on file with the NIH Office of Laboratory Animal Welfare, is registered with the United States Department of Agriculture (certificate #91-R-0001), and is accredited by Association for Assessment and Accreditation of Laboratory Animal Care (AAALAC) International of Frederick, MD.

Statistical Analysis. The data were expressed as means±standard deviation (SD). All statistical analyses were performed using GraphPad Prism 10.0 (GraphPad of Boston, MA). Statistical analysis was performed by using one-way analysis of variance (ANOVA) with Mann Whitney or Kruskal-Wallis post hoc tests. P values<0.05 were considered statistically significant (*P<0.05,**P<0.01,***P<0.001).

### RESULTS

Design of Oligonucleotide barcode and PCR amplification methodology. Oligobarcodes used for conjugation studies must be short enough for cellular uptake but also stable enough for Direct PCR amplification. Oligobarcoding of EVs occurred in a step-wise procedure: 1) EVs and oligobarcodes are separately functionalized to linker molecules, followed by 2) immediate incubation of newly functionalized EVs and oligobarcodes. Oligobarcodes were designed with 5′ amine groups to enable conjugation to sulfo-S-4FB linker molecules. Simultaneously, EVs were conjugated to sulfo-S-HyNic linker molecules as previously described, before covalent conjugation of both EVs and oligonucleotides using click chemistry (Zhang M, et al., ACS Applied Nano Materials. 2020;3(7):7211-22).

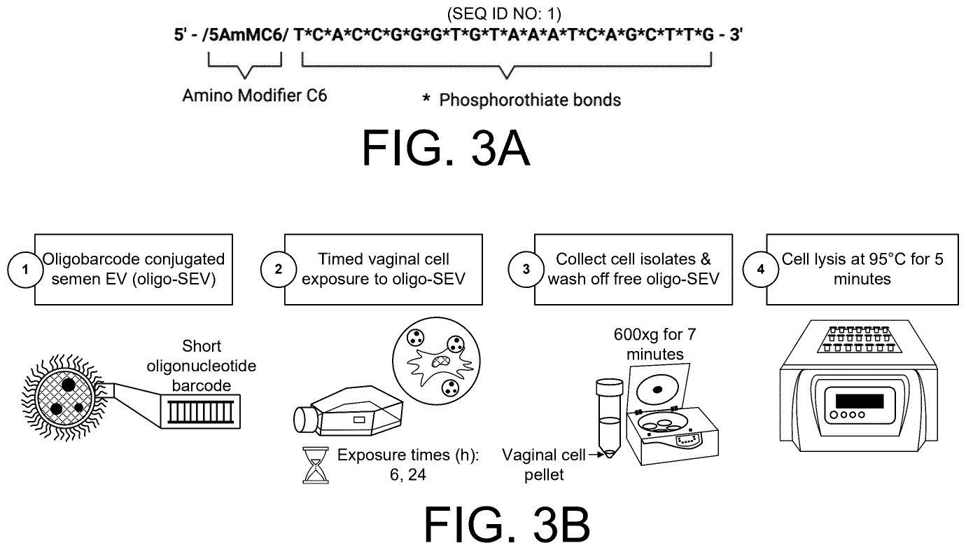

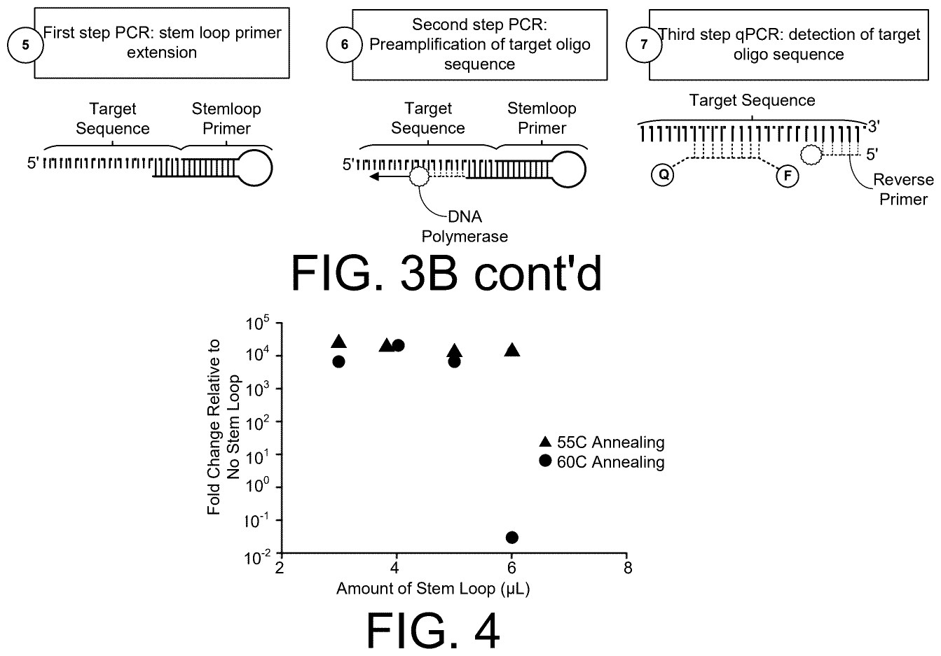

A barcode design that was 100-nucleotides long was tested for use in quantitative PCR (qPCR) reactions, but it was determined that long oligobarcodes resulted in high levels of background following qPCR amplification in control samples in this Example. Based on these results, a shorter 22-nucleotide barcode was tested. The 22-nucleotide barcode is based on the sequence of C. elegans microRNA cel-miR-39, of which commercial reagents using a stem-loop primer and PCR primer-probe sets are available. The short cel-miR-39 mimic barcode was further modified with phosphorthioate bonds to increase resistance to exonucleases (FIG. 3A). For detection of the microRNA, a stem-loop conversion reaction using a stem-loop primer was utilized, which is analogous to reverse-transcription (Schmittgen TD, et al., Methods. 2008;44(1):31-8; Kramer MF. Current Protocols in Molecular Biology. 2011;95(1):15). This stem-loop extended the short oligobarcodes with extra nucleotides to allow for the docking of primers to the target sequence for qPCR detection (FIG. 3B). Finally for qPCR, commercial cel-miR-39 primer-probe sets (Taqman miRNA assay) were used, which consist of two primers and a fluorescent fluorescein-based (FAM) probe to detect the target oligobarcode. Serial dilution experiments were performed to compare the expression level of oligobarcode detected with different amounts of stem-loop primer used in the reaction compared to a condition wherein no stem-loop primer is used at two different annealing temperatures. It was foung that using the stem-loop primer enabled oligobarcode detection by up to 104-fold greater than PBS controls and was robust across both dilutions of input stem-loop primer (FIG. 4).

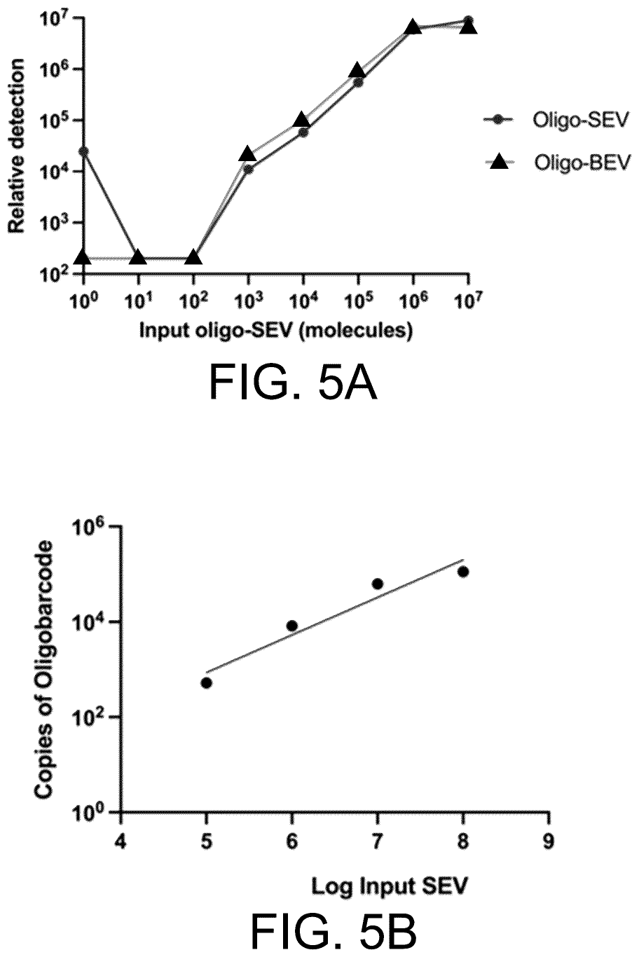

Oligo-EV conjugations were prepared using SEV and BEV, characterized oligo-EV conjugates using nanoparticle tracking analysis, then stem-loop conversion and qPCR were performed to detect the oligobarcode sequence on a dilution series of oligo-EV inputs. As little as 10 and as high as 10⁷ molecules of oligo-SEVs and oligo-BEVs were detected, demonstrating that this method can quantify the amount of barcode on a wide range of oligo-EV inputs (FIG. 5A). Digital PCR was also performed for absolute oligobarcode detection in oligo-SEV dilutions (FIG. 5B), which enabled estimation of the ratio of oligobarcode to EV and a detection limit for input number of oligo-EVs. Two copies of oligobarcode were detected per EV as quantified by digital PCR.

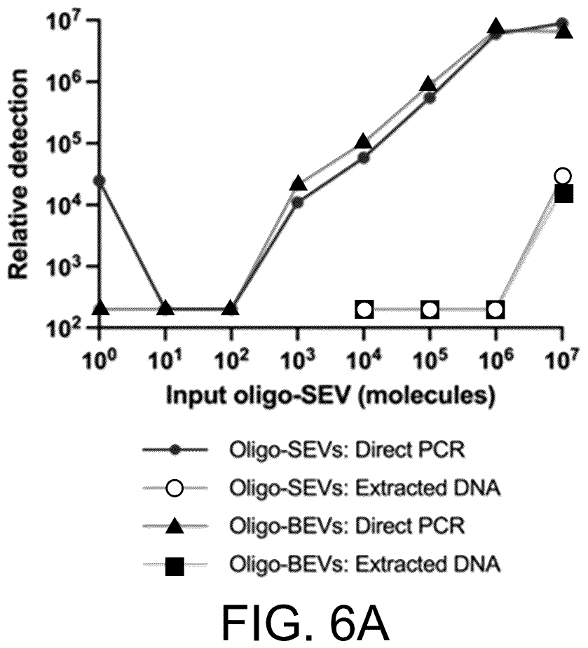

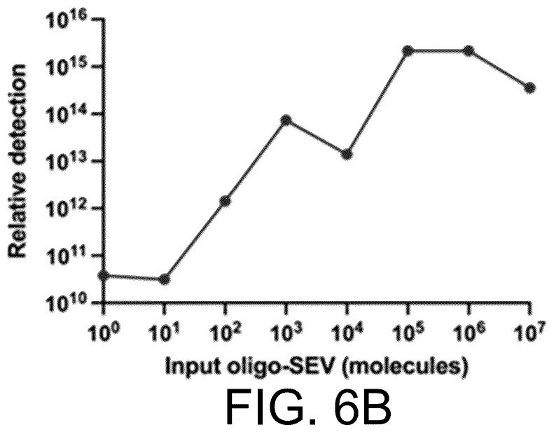

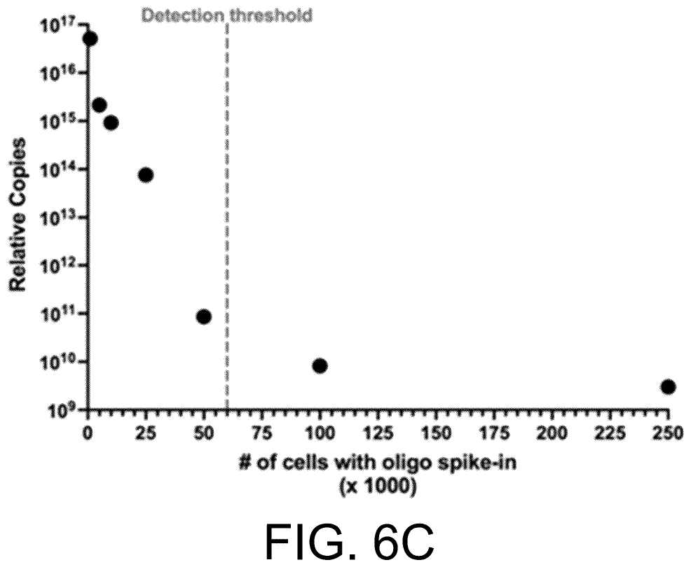

Application of oligobarcode-SEV in human in vivo THP cell culture and ex vivo vaginal tissue culture. A serial dilution series was run using oligo-SEV and oligo-BEV samples without cells to determine the limit of detection using qPCR in this Example. Though commercial DNA columns are typically used to isolate DNA from samples, there may be significant DNA oligobarcode loss that can occur due to the short length of the oligobarcodes (22 base pairs) combined with several column extraction steps required for the assay. To address this challenge, a Direct PCR approach was implemented to detect oligobarcodes in a one-step process, bypassing DNA columns. This approach was first tested without the use of cells (FIG. 6A) (Ben-Amar A, et al., 3 Biotech. 2017;7(4)). Rather than relying on DNA columns, Direct PCR used heat lysis to release the oligobarcodes into solution, which was then directly inputted into PCR for stem-loop extension. In this way, lengthy DNA extraction using separate columns was not required, but instead DNA can be directly extruded from EVs and cells in one heat lysis step. The signal of oligo-SEV detected in the samples had up to a 10,000-fold increase when Direct PCR was used compared to DNA extraction columns to detect oligobarcode (FIG. 6A). Oligo-SEV and oligo-BEV trends were similar, indicating uniform oligobarcode conjugation between both EV types and translatability of this technique.

Oligo-SEV conjugates were then applied to THP cells in vitro to test the detection limit and feasibility of oligobarcode detection in physiological environments. When applied to THP cell culture, it was found that oligobarcodes were detected in treated cells in a dose-dependent manner, notably for dosages greater than 100 molecules of oligo-SEV (FIG. 6B). Additionally, to investigate the relationship between cell number and detection of oligobarcode, seral dilutions of THP cells were exposed to a spike-in of 107 molecules of oligo-SEVs for 24h (FIG. 6C). From this experiment, oligobarcodes were detected in samples with as little as 1000 THP cells and in samples with up to 50,000 cells. There is a consistent inverse trend of decreased oligobarcode detection with increasing initial cell seeding density for the study. In this Example, no oligobarcode signal was detected in samples containing 100,000 and 250,000 cells.

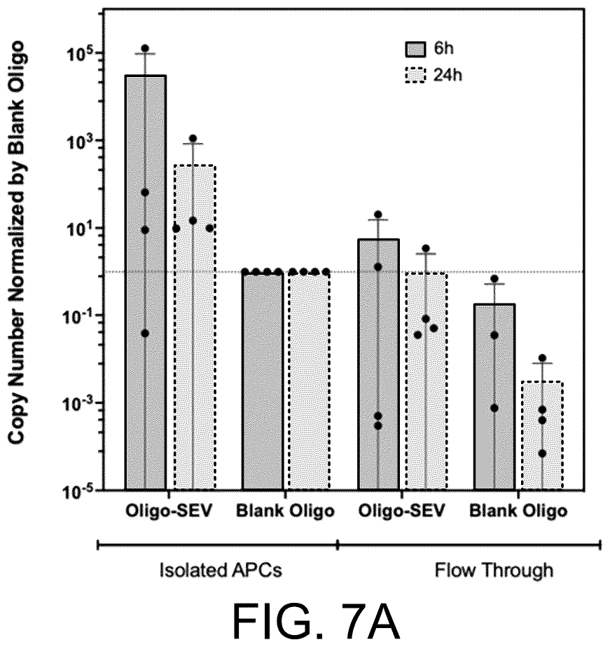

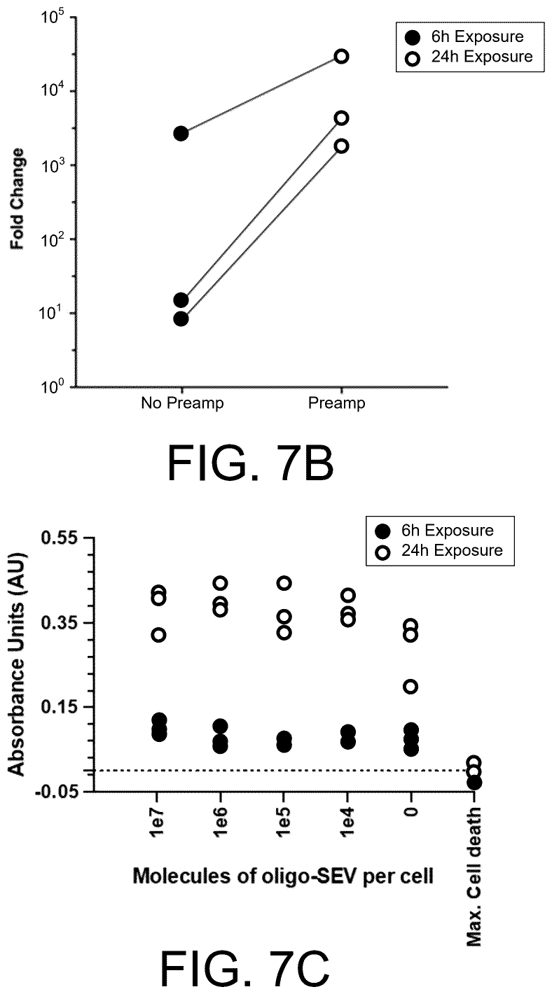

Following successful oligo-SEV detection in in vitro THP cells, oligo-SEVs were then administered to digested ex vivo human vaginal tissue. Oligo-SEVs and unconjugated oligobarcodes (blank oligo) were applied to digested vaginal tissue cultures at 106 molecules of oligo-SEV per cell for 6 and 24 hours. At each timepoint, cells were collected and washed to remove free oligo-SEVs. Magnetic cell separation based on positive selection of CD11c+ expressing cells was used to isolate the APCs from the remaining cells in the bulk sample (flow through). Oligobarcode uptake was measured and compared between the APCs and flow through cell populations using Direct and pre-amplified qPCR. Oligobarcode was detected in all samples exposed to oligo-SEVs for both 6 and 24 h timepoints. At these timepoints, oligo-SEV signal was detected in vaginal tissue up to 30,000-fold greater than unconjugated oligobarcodes, indicating that SEVs enhance the oligobarcode uptake within cells (FIG. 7A). It was also found that at both 6 and 24h timepoints oligo-SEVs were significantly localized with APCs compared to other cell types (flow through) by up to 100-fold, supporting their role as a first-responder to foreign entities within the body (FIG. 7A) (den Haan JM, et al., Immunol Lett. 2014;162(2 Pt B):103-12; Rodriguez-Pinto D. Cell Immunol. 2005;238(2):67-75; Wira CR, et al., Endocrinology. 2000;141(8):2877-85). This result is similar to previous data which used fluorescence-based tracking of SEV uptake to demonstrate that antigen-presenting cells (APCs) preferentially uptake SEV (Vojtech L, et al., PLOS ONE. 2019;14(10)). This result served as a proof of concept that the disclosed oligobarcodes can be detected when exposed to physiological conditions for up to 24 h post-EV exposure.

In some examples, stem-loop amplification can cause background amplification in samples when the initial stem-loop primer is not diluted enough in the subsequent PCR. However, dilution of the initial stem-loop product also reduced detection of oligobarcode in samples with low oligobarcode inputs and cellular DNA background. To reduce the background and improve the sensitivity for signal detection of oligobarcodes in vitro, a Taqman preamplification assay (Thermofisher) was used prior to qPCR. Taqmman preamplification (preamp) is 13-cycle PCR step that increased the amount of amplified target oligobarcode in the sample sufficiently above background noise. Ex vivo digested vaginal cells were treated with oligo-SEV for 6h, then processed with Direct PCR followed by the preamp step and qPCR. Results indicated that priming samples with preamp increased oligo-SEV detection by 10³-10⁴-fold compared to control samples without preamp (FIG. 7B).

To address potential concerns that the addition of engineered oligobarcodes with increased phosphorothioate bonds may cause toxicity to cells when exposed over long periods of time, cell viability assays were performed with ex vivo vaginal cells exposed to oligo-SEV. The cell viability assays showed no cytotoxicity following 6 h and 24 h of exposure to oligo-SEV, and cells remained viable and proliferative for up to 24 h of exposure to oligo-SEVs (FIG. 7C). Interestingly, cells demonstrated increased metabolic activity after 24 h compared to 6 h, suggesting proliferation even after oligo-SEV application.

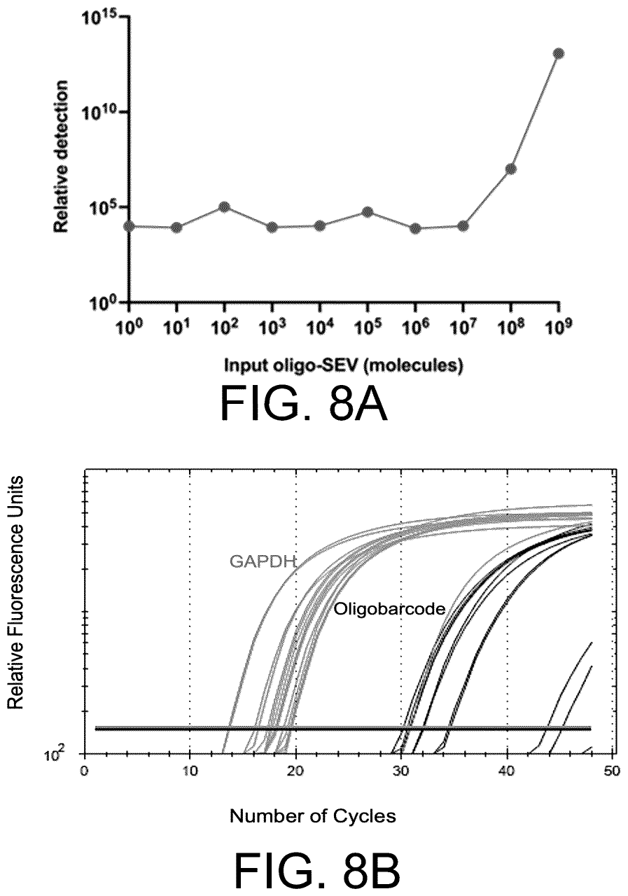

Application of oligo-BEVs in ex vivo organotypic whole hemisphere brain slices. One aspect of this innovative oligobarcoding technique using fast and efficient click chemistry conjugation is that it can be applied using EVs derived from any source. In addition to SEVs, oligobarcodes were conjugated to brain-tissue derived EVs (BEVs) and topically applied onto organotypic whole hemisphere brain slices. Leveraging a previously established isolation technique (Nguyen, supra), BEVs were derived from male whole neonatal rat brain tissue for this study. Different dilutions of oligobarcoded BEVs (oligo-BEVs) and blank oligo were topically applied to brain slices to demonstrate detection of oligobarcode in ex vivo brain tissue culture. Following 24h exposure of brain slices to oligo-BEVs, the brain tissue was collected, digested, and oligobarcode signals were detected using Direct and qPCR analyses. Oligobarcode signal was detected in brain slices exposed to oligo-BEVs in a dose-dependent manner, with noticeably more amplification detected after 108 molecules of EV administered to the sample (FIG. 8A). Oligobarcode signal was successfully detected in tissue slices at 215-217 fold greater than blank oligobarcodes when normalized by the housekeeper gene GAPDH (Glyceraldehyde 3-phosphate dehydrogenase) (FIG. 8B) (Derks NM, et al., Neuroscience. 2008;156(2):305-9; Al-Bader and Al-Sarraf. Brain Res Dev Brain Res. 2005;156(1):38-45). This indicates that oligo-BEVs are being actively uptaken by brain cells and are detectable following 24 h in tissue culture at physiological conditions. Furthermore, blank oligos were not detected in any sample regardless of the dilution factor used. These results demonstrate that oligobarcodes can be successfully conjugated to EVs from different sources and can be detected in cells both in vitro and ex vivo.

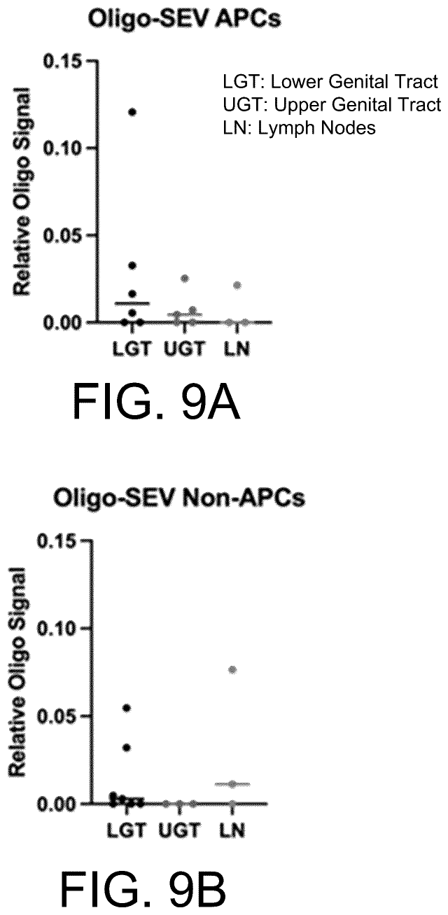

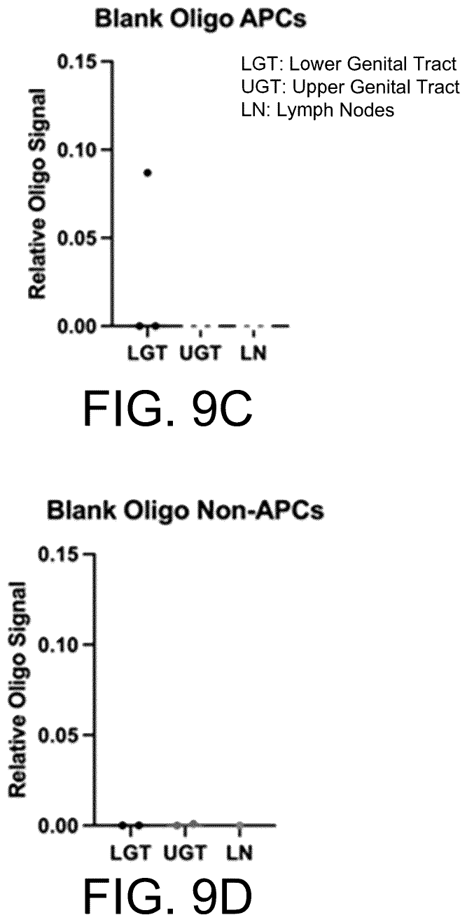

In vivo administration and qPCR detection of oligo-SEVs in murine models. Following the successful detection of oligobarcodes within both in vitro and ex vivo cultures, oligo-SEVs were applied to in vivo murine models. To test for the translatability of the oligobarcode technique for in vivo studies, one dose of oligo-SEVs were administered to female C57BL/6J mice. Genital tract and lymph node tissues were harvested 24 h after administration and immediately processed for analysis of oligobarcode signal in tissues with administered oligo-SEVs, blank unconjugated oligobarcodes, and saline controls (FIGS. 9A-9D). The tissues were digested, sorted, and Direct PCR and a probe-based qPCR were run on APCs and flow through cells as described for the in vitro studies. Overall, oligo-SEV detection levels were higher among APCs (FIGS. 9A, 9C) compared to non-APC flow through cells (FIGS. 9B, 9D) across all samples tested, indicating that oligo-SEVs may preferentially localize to APC cells. In the lower genital tract APCs expressed 133% greater oligo-SEV signal compared to flow through cells, and in the upper genital tract no oligo-SEVs were detected in the flow through at all. Blank oligobarcode was undetectable in almost every collected sample besides one APC sample from the lower genital tract, demonstrating that the SEVs are responsible for oligo-SEV uptake in cells compared to unconjugated oligobarcode (FIGS. 9C, 9D).

Of all APC fractions, those derived from the lower genital tract demonstrated the greatest oligobarcode expression change compared to a saline control followed by the upper genital tract and lymph nodes. In contrast, in the non-APC flow through cell fractions cells derived from the lymph nodes demonstrated greatest oligobarcode expression levels, though there is high variability in the samples analyzed for the lymph nodes.

### CONCLUSION

Existing labeling methods for EVs are non-specific, semi-quantitative, and often require expensive equipment and training which limits the ability to quantify EV localization within the body. The disclosed oligobarcoding technique addresses challenges posed by current labeling methods by providing a highly translatable, accessible, and quantitative method that can be used to label EVs from any source and can be used in vitro, ex vivo, and in vivo. Detection of oligobarcodes require accessible PCR instrumentation, which provides quantitative information about the degree of oligobarcode uptake within cells. This Example demonstrated that this oligobarcoding method is detectable across multiple biological models (in vitro, ex vivo, in vivo) using the disclosed multi-step PCR protocol, with a lower detection limit of 10 molecules of oligo-SEVs and a higher limit of 107 molecules of oligo-SEVs. In addition to testing oligo-SEVs, oligo-BEVs were also successfully detected using this proposed methodology. The oligo-EVs were detected in both vaginal and brain tissues using standard PCR techniques, which allow for greater translatability of this technology across different research spaces.

The oligobarcoding method disclosed herein is stable in physiological conditions up to 24h in different tissues. Oligobarcoding also did not induce cell cytotoxicity over this period of time, but rather, cells experienced increased metabolic activity likely due to proliferation between 6-24 h of oligo-SEV exposure. When applied in vitro, the upper limit of cells collected wherein oligo-SEVs signal remained detectable was 50,000. There was a noticeable decrease in oligo-SEV expression and number of cells seeded with the oligo-SEVs. Beyond the upper limit of 50,000 cells, Direct PCR may not work as well due to PCR inhibitors present in heat lysed cell samples. These PCR inhibitors increased as the number of initial cells seeded increased, resulting in decreased oligo-SEV signal expression. Oligo-SEVs were detected in vitro following 24 h of exposure to THP cells at various dilutions ranging from 1-10⁷ molecules of oligo-SEV in a generally dose-dependent manner.

Successful oligobarcode detection in vitro prompted further studies in more complex ex vivo physiological environments. To demonstrate the translatability of this technology, oligo-SEVs were applied to ex vivo human vaginal culture and oligo-BEVs were applied to ex vivo rat brain slices. When exposed to ex vivo vaginal tissues, oligo-SEV signals had significantly greater detection levels compared to blank unconjugated oligobarcodes, indicating that SEVs are involved in driving specific cellular uptake and without the SEVs blank oligobarcodes are not effectively trafficked by vaginal cells. Similarly, oligo-BEVs were also successfully detected following 24 h of exposure to ex vivo brain slices, whereas there was no signal from blank oligo.

To evaluate oligo-SEV uptake within specific cell populations, oligo-SEVs were applied to ex vivo vaginal cells for 24 h and APCS were immunoseparated from other cell populations (flow through). Oligo-SEVs are preferentially taken up by APCs in ex vivo vaginal cell culture compared to blank oligo, even after 6 h. Furthermore, APCs demonstrated greater uptake of oligo-SEVs compared to flow through cells at both 6 and 24 h timepoints. This demonstrates that not only are oligo-SEVs detectable after 24 h but they are also preferentially taken up by APCs compared to the flow through cells in vaginal tissue. As APCs are responsible for initiating immune response mechanisms against foreign microbes and pathogens, these results confirm that APCs uptake oligo-SEVs (den Haan, supra; Wira, supra).

Following the successful detection of oligobarcodes in two different ex vivo tissue models, oligo-SEVs were applied to in vivo murine models and cells were analyzed for the presence of oligobarcode signal. Oligo-SEV levels were detected in both APC and flow through cell populations, while blank unconjugated oligobarcodes were only detectable in one APC sample in the lower genital tract. These results indicate that APCs preferentially uptake oligo-SEVs compared to blank oligo across all organs analyzed. Furthermore, oligo-SEV signal was detected in APCs across all regions (lower genital tract, upper genital tract, lymph nodes). Specifically, the lower genital tract revealed the highest levels of oligo-SEV signal in this Example, followed by the lymph nodes and upper genital tract. This trend is not observed when evaluating oligo-SEV expression in non-APC flow through cells. As oligo-SEV intravaginal application occurred in the vagina, these results demonstrate that oligo-SEVs that were not completely trafficked to other organs after 24 h remained in the lower vaginal tract and interacted non-specifically to the cells in that region. In comparison, flow through cells exhibited the greatest level of oligo-SEV expression in the lower genital tract, but inconsistent levels of oligo-SEV expression in the lymph nodes and upper genital tract. These results demonstrate that the population of oligo-SEVs that remained in the lower genital tract did not get trafficked to other organs by non-APC flow through cells. Together, these results demonstrate that APCs may favor SEVs to uptake compared to other non-APC cells.

The ability to track EV uptake within cells is beneficial to advancing the EV research and therapeutics spaces. This Example describes the development and deployment of a novel oligonucleotide barcode tagging methodology for EVs using click chemistry. The disclosed results collectively demonstrate the translatability of this oligobarcoding technique to be detected from EVs across species and quantified with PCR to determine EV uptake within different cellular populations. Oligobarcodes address several concerns from existing EV labeling techniques by providing an accessible, low cost, sensitive, and quantitative detection methodology that can be readily applied to EVs obtained from any biological source.

### EXAMPLE CLAUSES

1. A method, including:
obtaining an extracellular vesicle;
attaching a first linker to the extracellular vesicle;
incubating the extracellular vesicle with an oligonucleotide label including a second linker, thereby conjugating the oligonucleotide label to the extracellular vesicle;
applying the conjugated extracellular vesicle to a subject or to a sample derived from the subject; and
detecting the presence of the conjugated extracellular vesicle in at least one cell of the subject.

2. The method of clause 1, wherein the first linker includes 4-formylbenzoate (4FB) and the second linker includes 6-hydrazinonicotinate acetone hydrazone (HyNic).

3. The method of clause 1 or 2, wherein the oligonucleotide label includes:
a primary amine conjugated to the second linker, and/or
at least one phosphorothioate bond.

4. The method of any of clauses 1-3, wherein a length of the oligonucleotide label is in a range of about 10 to about 80 nucleotides.

5. The method of any of clauses 1-4, wherein the extracellular vesicle and the oligonucleotide label are incubated at a ratio in a range of about 1:10 to about 1:100.

6. The method of any of clauses 1-4, wherein the extracellular vesicle and the oligonucleotide label are incubated at a ratio of about 1:80.

7. The method of any of clauses 1-6, wherein the detecting includes:
extracting the at least one cell from the subject; and/or
performing polymerase chain reaction (PCR), fluorescence in situ hybridization (FISH), DNA scope, RNA scope, isothermal amplification, or unbiased DNA sequencing.

8. An extracellular vesicle including an oligonucleotide label, the oligonucleotide label including at least one phosphorothioate bond.

9. The extracellular vesicle of clause 8, wherein the extracellular vesicle is derived from a living subject or in vitro cells.

10. The extracellular vesicle of clause 8 or 9, wherein a length of oligonucleotide label is in a range of about 10 to about 80 nucleotides, and/or
wherein the oligonucleotide label includes a primary amine.

11. A method, including:
administering a solution including the extracellular vesicle of any of clauses 8-10 to a living subject or a sample derived from the living subject; and
detecting the extracellular vesicle in at least one cell of the living subject.

12. The method of clause 11, wherein the living subject is a mouse, a rat, a mammal, a non-human primate, or human.

13. The method of clause 11 or 12, wherein the detecting includes at least one of:
extracting the at least one cell from the living subject;
lysing the at least one cell of the living subject; or
performing polymerase chain reaction (PCR), fluorescence in situ hybridization (FISH), DNA scope, RNA scope, isothermal amplification, or unbiased DNA sequencing.

14. The method of any of clauses 11-13, wherein the detecting includes examining the sample under a microscope, the method further including:
fixing the sample onto a slide.

15. The method of any of clauses 11-14, wherein the sample includes at least two cell types, the method further including:
detecting the extracellular vesicle in each of the at least two cell types.

16. A method, including:
analyzing PCR data from a sample; and
based on analyzing the PCR data, determining whether the extracellular vesicle of any of clauses 8-10 is present in the sample.

17. A kit, including:
an oligonucleotide barcode including a first linker;
a second linker; and
reagents configured to conjugate the second linker to an extracellular vesicle.

18. The kit of clause 17, wherein the first linker includes 4-formylbenzoate (4FB) and the second linker includes 6-hydrazinonicotinate acetone hydrazone (HyNic).

19. The kit of clause 17 or 18, wherein the reagents are for covalent conjugation, lipofection, or electroporation.

20. The kit of any of clauses 17-19, further including:
reagents for purification of extracellular vesicles from a tissue sample; and/or polymerase chain reaction (PCR) reagents for detection of the oligonucleotide barcode.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be used for realizing implementations of the disclosure in diverse forms thereof.

As will be understood by one of ordinary skill in the art, each implementation disclosed herein can comprise, consist essentially of or consist of its particular stated element, step, or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the implementation to the specified elements, steps, ingredients or components and to those that do not materially affect the implementation. As used herein, the term “based on” is equivalent to “based at least partly on,” unless otherwise specified.

Unless otherwise indicated, all numbers expressing quantities, properties, conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing implementations (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate implementations of the disclosure and does not pose a limitation on the scope of the disclosure. No language in the specification should be construed as indicating any non-claimed element essential to the practice of implementations of the disclosure.

Groupings of alternative elements or implementations disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Variants of the sequences disclosed and referenced herein are also included. Variants of the protein, nucleic acid, and gene sequences disclosed herein also include sequences with at least 70% sequence identity, 80% sequence identity, 85% sequence, 90% sequence identity, 95% sequence identity, 96% sequence identity, 97% sequence identity, 98% sequence identity, or 99% sequence identity to the protein, nucleic acid, or gene sequences disclosed herein.

“% sequence identity” refers to a relationship between two or more sequences, as determined by comparing the sequences. In the art, “identity” also means the degree of sequence relatedness between protein, nucleic acid, or gene sequences as determined by the match between strings of such sequences. “Identity” (often referred to as “similarity”) can be readily calculated by known methods, including those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul, et al., J. Mol. Biol. 215:403-410 (1990); DNASTAR (DNASTAR, Inc., Madison, Wisconsin); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y. Within the context of this disclosure it will be understood that where sequence analysis software is used for analysis, the results of the analysis are based on the “default values” of the program referenced. As used herein “default values” will mean any set of values or parameters, which originally load with the software when first initialized.

Variants also include nucleic acid molecules that hybridizes under stringent hybridization conditions to a sequence disclosed herein and provide the same function as the reference sequence. Exemplary stringent hybridization conditions include an overnight incubation at 42° C. in a solution including 50% formamide, 5XSSC (750 mM NaCl, 75 mM trisodium citrate), 50 mM sodium phosphate (pH 7.6), 5XDenhardt′s solution, 10% dextran sulfate, and 20 μg/ml denatured, sheared salmon sperm DNA, followed by washing the filters in 0.1XSSC at 50° C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37°° C. in a solution including 6XSSPE (20XSSPE=3M NaCl; 0.2M NaH2PO4; 0.02M EDTA, pH 7.4), 0.5% SDS, 30% formamide, 100 μg/ml salmon sperm blocking DNA; followed by washes at 50° C. with 1XSSPE, 0.1% SDS. In addition, to achieve even lower stringency, washes performed following stringent hybridization can be done at higher salt concentrations (e.g. 5XSSC). Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

“Specifically binds” refers to an association of a binding domain (of, for example, a CAR binding domain or a nanoparticle selected cell targeting ligand) to its cognate binding molecule with an affinity or Ka (i.e., an equilibrium association constant of a particular binding interaction with units of 1/M) equal to or greater than 10⁵ M⁻¹, while not significantly associating with any other molecules or components in a relevant environment sample. “Specifically binds” is also referred to as “binds” herein. Binding domains may be classified as “high affinity” or “low affinity”. In particular embodiments, “high affinity” binding domains refer to those binding domains with a Ka of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹. In particular embodiments, “low affinity” binding domains refer to those binding domains with a Ka of up to 10⁷ M⁻¹, up to 10⁶ M⁻¹, up to 10⁵ M⁻¹. Alternatively, affinity may be defined as an equilibrium dissociation constant (Kd) of a particular binding interaction with units of M (e.g., 10⁻⁵ M to 10⁻M). In certain embodiments, a binding domain may have “enhanced affinity,” which refers to a selected or engineered binding domains with stronger binding to a cognate binding molecule than a wild type (or parent) binding domain. For example, enhanced affinity may be due to a Ka (equilibrium association constant) for the cognate binding molecule that is higher than the reference binding domain or due to a Kd (dissociation constant) for the cognate binding molecule that is less than that of the reference binding domain, or due to an off-rate (Koff) for the cognate binding molecule that is less than that of the reference binding domain. A variety of assays are known for detecting binding domains that specifically bind a particular cognate binding molecule as well as determining binding affinities, such as Western blot, ELISA, and BIACORE® analysis (see also, e.g., Scatchard, et al., 1949, Ann. N.Y. Acad. Sci. 51:660; and U.S. Pat. Nos. 5,283,173, 5,468,614, or the equivalent).

Unless otherwise indicated, the practice of the present disclosure can employ conventional techniques of immunology, molecular biology, microbiology, cell biology and recombinant DNA. These methods are described in the following publications. See, e.g., Green and Sambrook, Molecular Cloning: A Laboratory Manual, 4nd Edition (2012); F. M. Ausubel, et al. eds., Current Protocols in Molecular Biology, (2003); the series Methods In Enzymology (Academic Press, Inc.); Behlke, et al., Polymerase Chain Reaction: Theory and Technology (2019); Greenfield, ed. Antibodies, A Laboratory Manual, Second Edition (2014); and Capes-Davis and R. I. Freshney, eds. Freshney's Culture of Animal Cells 8th Edition (2021).

Certain implementations are described herein, including the best mode known to the inventors for carrying out implementations of the disclosure. Of course, variations on these described implementations will become apparent to those of ordinary skill in the art upon reading the foregoing description. The inventor expects skilled artisans to employ such variations as appropriate, and the inventors intend for implementations to be practiced otherwise than specifically described herein. Accordingly, the scope of this disclosure includes all modifications and equivalents of the subject matter recited in the claims appended hereto as permitted by applicable law. Moreover, any combination of the above-described elements in all possible variations thereof is encompassed by implementations of the disclosure unless otherwise indicated herein or otherwise clearly contradicted by context.

1. A method, comprising:
obtaining an extracellular vesicle;
attaching a first linker to the extracellular vesicle;
incubating the extracellular vesicle with an oligonucleotide label comprising a second linker, thereby conjugating the oligonucleotide label to the extracellular vesicle;
applying the conjugated extracellular vesicle to a subject or to a sample derived from the subject; and
detecting the presence of the conjugated extracellular vesicle in at least one cell of the subject.

2. The method of claim 1, wherein the first linker comprises 4-formylbenzoate (4FB) and the second linker comprises 6-hydrazinonicotinate acetone hydrazone (HyNic).

3. The method of claim 1, wherein the oligonucleotide label comprises:
a primary amine conjugated to the second linker, and/or
at least one phosphorothioate bond.

4. The method of claim 1, wherein a length of the oligonucleotide label is in a range of about 10 to about 80 nucleotides.

5. The method of claim 1, wherein the extracellular vesicle and the oligonucleotide label are incubated at a ratio in a range of about 1:10 to about 1:100.

6. The method of claim 1, wherein the extracellular vesicle and the oligonucleotide label are incubated at a ratio of about 1:80.

7. The method of claim 1, wherein the detecting comprises:
extracting the at least one cell from the subject; and/or
performing polymerase chain reaction (PCR), fluorescence in situ hybridization (FISH), DNA scope, RNA scope, isothermal amplification, or unbiased DNA sequencing.

8. An extracellular vesicle comprising an oligonucleotide label, the oligonucleotide label comprising at least one phosphorothioate bond.

9. The extracellular vesicle of claim 8, wherein the extracellular vesicle is derived from a living subject or in vitro cells.

10. The extracellular vesicle of claim 8, wherein a length of oligonucleotide label is in a range of about 10 to about 80 nucleotides, and/or
wherein the oligonucleotide label comprises a primary amine.

11. A method, comprising:
administering a solution comprising the extracellular vesicle of claim 8 to a living subject or a sample derived from the living subject; and
detecting the extracellular vesicle in at least one cell of the living subject.

12. The method of claim 11, wherein the living subject is a mouse, a rat, a mammal, a non-human primate, or human.

13. The method of claim 11, wherein the detecting comprises at least one of:
extracting the at least one cell from the living subject;
lysing the at least one cell of the living subject; or
performing polymerase chain reaction (PCR), fluorescence in situ hybridization (FISH), DNA scope, RNA scope, isothermal amplification, or unbiased DNA sequencing.

14. The method of claim 11, wherein the detecting comprises examining the sample under a microscope, the method further comprising:
fixing the sample onto a slide.

15. The method of claim 11, wherein the sample comprises at least two cell types, the method further comprising:
detecting the extracellular vesicle in each of the at least two cell types.

16. A method, comprising:
analyzing PCR data from a sample; and
based on analyzing the PCR data, determining whether the extracellular vesicle of claim 8 is present in the sample.

17. A kit, comprising:
an oligonucleotide barcode comprising a first linker;
a second linker; and
reagents configured to conjugate the second linker to an extracellular vesicle.

18. The kit of claim 17, wherein the first linker comprises 4-formylbenzoate (4FB) and the second linker comprises 6-hydrazinonicotinate acetone hydrazone (HyNic).

19. The kit of claim 17, wherein the reagents are for covalent conjugation, lipofection, or electroporation.

20. The kit of claim 17, further comprising:
reagents for purification of extracellular vesicles from a tissue sample; and/or
polymerase chain reaction (PCR) reagents for detection of the oligonucleotide barcode.
